(19) United States
(12) Patent Application Publication
(10) Pub. No.: US 2005/0176654 A1
(43) Pub. Date: Aug. 11, 2005

(54) Composition for regulating bone metabolism

(75) Inventors: Kinya Takagaki, Fukuoka (JP); Takeshi Mitsui, Fukuoka (JP)

Correspondence Address: AMIN & TUROCY, LLP, 1900 EAST 9TH STREET, NATIONAL CITY CENTER, 24TH FLOOR,, CLEVELAND, OH, 44114, US

(73) Assignee: Toyo Shinyaku Co., Ltd.

(21) Appl. No.: 11/020,435

(22) Filed: Dec. 22, 2004

(30) Foreign Application Priority Data
Feb. 28, 2003 (JP) 2003-054076

(51) Int. Cl. A61K 31/7048; A61K 31/353; A61K 35/78

(52) U.S. Cl. 514/27; 514/456; 424/725

(57) ABSTRACT

The present invention provides a composition for regulating bone metabolism that comprises proanthocyanidins. It is preferable that the proanthocyanidins in this composition for regulating bone metabolism are contained in an extract derived from a natural product, and the extract comprises at least 20 wt % of oligomeric proanthocyanidins having a degree of polymerization of 2 to 4 in terms of dry weight. The composition of the present invention provides an effect of regulating bone metabolism.

### BACKGROUND OF THE INVENTION

1. Field of the Invention

The present invention relates to a composition providing an effect of regulating bone metabolism.

2. Description of the Related Art

Osteoporosis is a symptom in which bones become fragile due to a reduction in bone mass (bone mineral density) and is common among middle-aged and older people, in particular, postmenopausal women. Bones are renewed through a process in which formation of new bone, known as osteogenesis, and destruction of old bone, known as bone resorption, are repeated, with the balance of the metabolism being maintained. However, when this balance is disturbed and bone resorption becomes dominant, bones are destructed slowly, and bones become spongy, and thus become fragile. The balance between osteogenesis and bone resorption is disturbed due to, for example, a decrease in absorption of calcium, a decrease in active vitamin D, and the like in the case of middle-aged and older people, and estrogen deficiency and the like in the case of postmenopausal women.

Osteogenesis is performed by the following process: First, osteoblasts secrete collagen and bone proteins containing γ-calboxyglutamic acid (i.e., osteocalcin; hereinafter referred to as OC) that is a protein having glutamic acid residues that are carboxylated in a vitamin K-dependent manner, and then, calcium phosphate deposits on these proteins (namely, bone mineral is formed).

In bone resorption, osteoclasts decomposes the above-mentioned proteins and bone mineral.

Such balance between osteogenesis and bone resorption can be evaluated employing OC in blood as an indicator. More specifically, when osteogenesis occurs, active OC in which glutamic acid residues have a higher degree of calboxylation is increased in blood, and when bone resorption by osteoclasts occurs, inactive OC in which glutamic acid residues are not calboxylated or have a lower degree of calboxylation is released in blood. For example, in an experiment using rats, the ratio of quantity of the active OC to that of the inactive OC in blood stays constant when the rats are in a healthy state. However, in the case of model rats that were spayed so that bone resorption is greater than osteogenesis and the rats are in the postmenopausal state, this ratio of quantities is low.

In recent years, the society is facing an aging population, and it is presumed that diseases caused by such decrease in bones will increase more and more, which is a problem to be overcome. In particular, it is known that even in young people, decrease in bone mass, so-called “juvenile osteoporosis”, is increasing with a rapid change in eating habits.

Presently, various foods and pharmaceuticals using materials for preventing bone diseases have been proposed. Examples thereof include foods or drugs containing a female hormone-like substance, a component that is necessary for osteogenesis, a component having an ability of inhibiting bone resorption, or the like. Please refer to e.g., Japanese Patent No. 3250071 and Japanese Laid-Open Patent Publication Nos. 2000-247896, 2000-191542, and 7-144018. However, all of such foods and drugs merely prevent the development of osteoporosis to some extent, and no methods for preventing and treating bone diseases by regulating the balance between osteogenesis and bone resorption, which is involved in a fundamental cause of bone diseases, to a healthy condition have been reported.

Therefore, there is a demand for a food or a drug that can regulate bone metabolism.

### SUMMARY OF THE INVENTION

As a result of in-depth research on components derived from natural products for regulating the balance between osteogenesis and bone resorption, the inventors of the present invention found that a composition comprising proanthocyanidins provides an effect of regulating bone metabolism, and thus achieved the present invention.

The present invention provides a composition for regulating bone metabolism that comprises a proanthocyanidin. In a preferred embodiment, the proanthocyanidin is contained in an extract derived from a natural product, and the extract comprises at least 20 wt % of oligomeric proanthocyanidins having a degree of polymerization of 2 to 4 in terms of dry weight.

According to the present invention, through the ingestion of the composition for regulating bone metabolism that contains proanthocyanidins, osteogenesis can be promoted, and the balance of bone metabolism can be restored to a healthy condition. This composition can be used for health food products or pharmaceuticals for preventing osteoporosis or improving the conditions of osteoporosis and the like.

### BRIEF DESCRIPTION OF THE DRAWINGS

FIG. 1 is a graph showing an active OC content over time when an OPC-containing aqueous solution or water was administered to rats for 63 days.

FIG. 2 is a graph showing an inactive OC content over time when the OPC-containing aqueous solution or the water was administered to the rats for 63 days.

FIG. 3 is a graph showing a ratio of active OC content/inactive OC content over time when the OPC-containing aqueous solution or the water was administered to the rats for 63 days.

### DESCRIPTION OF THE PREFERRED EMBODIMENT

Hereinafter, the composition for regulating bone metabolism of the present invention will be described. It should be noted that the following description is not limiting the present invention, and it is apparent to those skilled in the art that various alternations can be made within the scope of the spirit of the present invention.

In the present invention, proanthocyanidins refer to a group of compounds that are condensation products having flavan-3-ol and/or flavan-3,4-diol as a constituent unit and having a degree of polymerization of 2 or more.

As the proanthocyanidins, proanthocyanidins containing a large amount of condensation products having a lower degree of polymerization are preferably used. As such condensation products, condensation products having a degree of polymerization of 2 to 30 (dimer to 30-mer) are preferable, condensation products having a degree of polymerization of 2 to 10 (dimer to decamer) are more preferable, and condensation products having a degree of polymerization of 2 to 4 (dimer to tetramer) are even more preferable. In this specification, the condensation products having a degree of polymerization of 2 to 4 are referred to as oligomeric proanthocyanidins (OPCs). Proanthocyanidins, which are one type of polyphenol, are potent antioxidants produced by plants, and contained concentratedly in portions of plant leaves, bark, or skin or seeds of fruits. More specifically, proanthocyanidins, in particular, OPCs are contained in the bark of pine, oak, bayberry, and the like; the fruit or seeds of grape, blueberry, strawberry, avocado, locust, and cowberry; the hull of barley, wheat, soybean, black soybean, cacao, adzuki bean, and conker; the inner skin of peanuts; and the leaves of ginkgo, for example. Moreover, it is known that OPCs are also contained in cola nuts in West Africa; the roots of Rathania in Peru; and Japanese green tea. OPCs are substances that cannot be produced in the human body.

For the proanthocyanidins contained in the composition for regulating bone metabolism of the present invention, foodstuff raw materials such as ground products or extracts from the above-mentioned barks, or fruits or seeds can be used. In particular, it is preferable to use a pine bark extract. Among proanthocyanidins, OPCs are especially abundant in pine bark, and thus, a pine bark extract is preferably used as a raw material of the proanthocyanidins.

Hereinafter, a method for preparing proanthocyanidins will be described taking a pine bark extract that contains OPCs abundantly as an example.

As the pine bark extract, an extract from the bark of plant belonging to Pinales, such as French maritime pine (Pinus martima), Larix leptolepis, Pinus thunbergii, Pinus densiflora, Pinus parviflora, Pinus pentaphylla, Pinus koraiensis, Pinus pumila, Pinus luchuensis, utsukushimatsu (Pinus densiflora form. umbraculifera), Pinus palustris, Pinus bungeana, and Anneda in Quebec, Canada, can be preferably used. Among these, French maritime pine (Pinus martima) bark extract is preferable.

French maritime pine refers to maritime pines that grow in a part of the Atlantic coastal area in southern France. It is known that the bark of this French maritime pine contains proanthocyanidins, organic acids, and other bioactive substances, and proanthocyanidins, which are the main component of the French maritime pine bark, are known to have a potent antioxidation ability of removing active oxygen.

The pine bark extract is obtained by extracting the bark of the above-mentioned pines using water or an organic solvent. When water is used, it is preferable to employ warm water or hot water. The water may contain a salt such as sodium chloride. As the organic solvent that can be employed for extraction, an organic solvent that is acceptable for production of foods or pharmaceuticals can be employed. Examples of such solvent include methanol, ethanol, 1-propanol, 2-propanol, 1-butanol, 2-butanol, acetone, hexane, cyclohexane, propylene glycol, aqueous ethanol, aqueous propylene glycol, methyl ethyl ketone, glycerin, methyl acetate, ethyl acetate, diethyl ether, dichloromethane, edible oils or fats, 1,1,1,2-tetrafluoroethane, and 1,1,2-trichloroethene. The water and the organic solvents may be used alone or in combination. In particular, hot water, aqueous ethanol, and aqueous propylene glycol are preferably used.

The method for extracting proanthocyanidins from pine bark is not particularly limited, and heat extraction or supercritical fluid extraction can be employed, for example.

Supercritical fluid extraction is a method for performing extraction using a supercritical fluid. A supercritical fluid is in a state that is above the liquid-vapor critical point in the phase diagram showing critical temperature and critical pressure. Examples of compounds that can be employed as a supercritical fluid include carbon dioxide, ethylene, propane, and nitrous oxide (laughter gas). Carbon dioxide is preferably used.

Supercritical fluid extraction includes an extraction step in which a target component is extracted with a supercritical fluid and a separation step in which the target component is separated from the supercritical fluid. In the separation step, any separation process can be employed, examples of which include a separation based on a change in pressure, a separation based on a change in temperature, and a separation using an adsorbent or absorbent.

Moreover, it is also possible to perform supercritical fluid extraction in which an entrainer is added. In this method, extraction is performed using an extracting fluid obtained by adding, for example, ethanol, propanol, n-hexane, acetone, toluene, or another aliphatic lower alcohol, aliphatic hydrocarbon, aromatic hydrocarbon, or ketone at about 2 to 20 W/V % to a supercritical fluid, so that the solubility of a target substance to be extracted, such as OPCs and catechins (described later), in the extracting fluid is dramatically increased or the selectivity of separation is enhanced. Thus, a pine bark extract is obtained efficiently.

Since supercritical fluid extraction can be performed at a relatively low temperature, it has the following advantages: it is applicable for extracting substances that deteriorate or decompose at high temperatures; the extracting fluid does not remain; and the extracting fluid can be recovered and recycled, so that a step of removing the extracting fluid and the like can be omitted, and thus, the process can be simplified.

Furthermore, methods other than those mentioned above can be employed for extraction from pine bark, and the examples of which include a batch method using liquid carbon dioxide, a reflux method using liquid carbon dioxide, and a reflux method using supercritical carbon dioxide, and the like.

It is also possible to employ a combination of a plurality of extraction processes to perform extraction from pine bark. By combining a plurality of extraction processes, pine bark extracts with various components can be obtained.

In the present invention, the pine bark extract that contains proanthocyanidins as the main component is specifically prepared using the following method. However, this method is merely an example, and the present invention is not limited to this method.

First, 1 kg of the bark of French maritime pine is immersed in 3 L of a saturated solution of sodium chloride, and extraction is performed for 30 minutes at 100° C. to obtain an extract liquid (extraction step). Then, the extract liquid is filtrated, and the resultant insoluble material is washed with 500 ml of a saturated solution of sodium chloride to obtain a washed liquid (washing step). The extract liquid and the washed liquid are combined to obtain a crude extract liquid of pine bark.

Next, 250 ml of ethyl acetate is added to this crude extract liquid, mixed, and separated to obtain an ethyl acetate layer. This process is repeated five times, and the obtained ethyl acetate layers are combined. The resultant ethyl acetate extract is added directly to 200 g of anhydrous sodium sulfate for drying. Then, this ethyl acetate extract is filtrated, and the filtrated extract is concentrated under a reduced pressure to a volume of ⅕ of the original filtrated extract. The concentrated ethyl acetate extract is poured into 2 L of chloroform and stirred, and the resultant precipitate is recovered by filtration. Subsequently, this precipitate is dissolved in 100 ml of ethyl acetate, and then the resultant solution is added to 1 L of chloroform to form a precipitate. This process is repeated twice, and thus, a washing process is accomplished. With this method, for example, about 5 g of pine bark extract containing at least 20 wt % of OPCs that have a degree of polymerization of 2 to 4 and at least 5 wt % of catechins can be obtained.

The extract derived from a raw material plant such as the above-mentioned pine bark contains preferably at least 20 wt % of OPCs and more preferably at least 30 wt % of OPCs in terms of dry weight. As such raw material having a higher proanthocyanidin content, a pine bark extract can be preferably used.

It is preferable that the above-mentioned raw material plant extract contains at least 5 wt % of catechins as well as proantocyanidins, in particular, OPCs. The term “catechins” is a general term referring to polyhydroxyflavan-3-ols. As the catechins, for example, (+)-catechin, (−)-epicatechin, (+)-gallocatechin, (−)-epigallocatechin, epigallocatechin gallate, and epicatechin gallate are known. From extracts derived from raw material plants such as the above-mentioned pine bark, gallocatechin, afzelechin, and 3-galloyl derivatives of (+)-catechin or gallocatechin are isolated in addition to (+)-catechin that is called catechin in a narrow sense. Catechins are known to have a cancer inhibiting ability, an arteriosclerosis preventing ability, a lipid metabolism disorder inhibiting ability, a blood pressure elevation inhibiting ability, a platelet aggregation inhibiting ability, an antiallergic ability, an antiviral ability, an antibacterial ability, a dental caries preventing ability, a halitosis preventing ability, an intestinal flora normalization ability, an active oxygen or free radical eliminating ability, an antioxidation ability, and the like. Moreover, catechins are known to have an antidiabetic ability that inhibits an elevation of blood glucose. Catechins have the property of both increasing the solubility in water and being activated in the presence of OPCs. Therefore, catechins enhance the abilities of OPCs when ingested together with OPCs.

It is preferable that catechins are contained in the above-mentioned raw material plant extracts in a ratio of 5 wt % or more. Alternatively, it is also preferable that a formulation is prepared so that it contains a raw material plant extract containing at least 20 wt % of OPCs and furthermore, contains catechins in a ratio of 5 wt % or more. For example, when the catechin content in a pine bark extract is less than 5 wt %, it is possible to add catechins so that the catechin content becomes at least 5 wt %. It is most preferable to use a pine bark extract containing at least 5 wt % of catechins and at least 20 wt % of OPCs.

Proanthocyanidins can provide an effect of regulating the balance between osteogenesis and bone resorption. In particular, they can provide an effect of regulating this balance that has been disturbed due to menopausal disorders to a healthy condition. The mechanism of this regulation of bone metabolism is unclear, but it seems that osteogenesis is promoted. This is apparent from the fact that at least the osteogenesis marker (i.e., active OC) in blood increases.

In particular, when proanthocyanidins having a higher OPC content or an extract containing proanthocyanidins having a higher OPC content is used, a better effect of regulating bone metabolism can be achieved than in the case where proanthocyanidins having a higher degree of polymerization (having a lower OPC content) are used.

It is known that since proanthocyanidins, in particular, OPCs are antioxidants as mentioned above, they also provide an effect of reducing the possibility of adult diseases, such as cancer and cardiac diseases, an effect of improving allergic diathesis, such as arthritis, atopic dermatitis, and pollenosis, an effect of inhibiting oxidation and degradation of collagen, and the like. Among these, especially the effect of proanthocyanidins of inhibiting oxidation and degradation of collagen contributes to preventing a reduction in bone mass effectively because collagen is the main protein that constitutes the bone.

The composition for regulating bone metabolism of the present invention comprises proanthocyanidins in a ratio of preferably at least 0.1 wt % and more preferably at least 1 wt % in terms of dry weight. It is preferable that the composition especially contains a large amount of OPCs, and the OPC content in the composition is preferably at least 0.02 wt % and more preferably at least 0.2 wt % in terms of dry weight. This composition can be used for foods, pharmaceuticals, and the like.

Moreover, the composition of the present invention may comprise ascorbic acid or a derivative thereof in order to allow OPCs to exert their effects more efficiently. It is known that when ingested together with OPCs, ascorbic acid works synergistically with OPCs so that the stability of the two components is increased, and furthermore, the absorptivity and the persistence of bioactivity of the ascorbic acid are increased. Since the OPCs that are contained in the composition of the present invention have an ability of promoting synthesis of collagen, which is a constituent protein of the bone, the OPCs can promote osteogenesis when they are ingested together with ascorbic acid.

Examples of ascorbic acid and its derivatives that can be contained in the composition of the present invention include food additives, such as ascorbyl glycoside, sodium ascorbate, and magnesium ascorbate, and natural materials that contain ascorbic acid abundantly. Examples of such natural materials include natural materials derived from fruits such as lemon, orange, acerola, and the like and natural materials derived from vegetables such as broccoli, Brussels sprouts, pimento, Brassica campestris, and cauliflower.

The weight ratio of proanthocyanidines and ascorbic acid or derivative thereof is preferably in the range of 1:0.1 to 1:50 and more preferably 1:0.2 to 1:20.

The composition for regulating bone metabolism of the present invention may contain additives, such as excipients, extenders, binders, thickeners, emulsifiers, lubricants, humectants, suspending agents, coloring agents, flavors, food additives, and seasonings, if necessary. Examples of food additives include nutritions, such as royal jelly, vitamins, proteins, calcium substances such as eggshell calcium, lecithin, chlorella powder, Angelica keiskei powder, and molokheiya powder. Examples of seasonings include stevia powder, ground green tea powder, lemon powder, honey, maltitol, lactose, and sugar solutions. Each components of the composition can be made into the form of capsules such as hard capsules and soft capsules, tablets, or pills, or they can be made into the form of powder, granule, tea bags, candy, liquid, paste, or the like. According to the form of the composition or according to preference, the composition may be eaten or drunk as it is, or may be dissolved in water, hot water, milk, or the like and drunk.

Although there is no limitation regarding the daily intake amount of the composition for regulating bone metabolism of the present invention, it is preferable that the amount of the proanthocyanidins is within the range of 0.02 g to 1 g. The amount of the ascorbic acid or derivative thereof that is suitable for the amount of the proanthocyanidins within this range is preferably 0.1 g to 1 g.

When a suitable amount of the composition for regulating bone metabolism of the present invention is ingested, the composition provides the effect of promoting osteogenesis and provides the effect of regulating the balance of bone metabolism between osteogenesis and bone resorption to a healthy condition. Furthermore, when an extract containing at least 20 wt % of OPCs in terms of dry weight are employed for proanthocyanidins, a particularly excellent effect of regulating bone metabolism can be achieved. In this way, the composition of the present invention is effective in improving bone disorders, in particular, osteoporosis.

### EXAMPLES

Hereinafter, the present invention will be described by way of examples. However, the present invention is not limited to these examples.

Preparation of Model Rats

First, 15 SD rats in a group of female SD rats (Charles River Japan, Inc.) at the age of nine weeks were spayed surgically. The remaining 10 SD rats underwent a pseudo operation. All of the SD rats were given a standard feed (MF feed for mouse, rat, and hamster: Oriental Yeast Co., Ltd.) and water for three weeks for acclimation. Then, the pseudo-operated rats were divided into two groups of 5 each so that the average weight was almost equal between the groups. Furthermore, the spayed rats were divided into three groups of 5 each so that the average weight in each group is almost equal to the average weight in the pseudo operation groups.

### Example 1

First, an OPC-containing aqueous solution in which a pine bark extract (trade name: Flavangenol, produced by TOYO SHINYAKU Co., Ltd.) was contained in a ratio of 2.5 mg/mL was prepared. The pine bark extract contained 5 wt % of OPCs in terms of dry weight. Then, 1 mL/kg body weight of the OPC-containing aqueous solution was orally administered to each of 5 spayed rats in one of the groups using a sonde once a day for 63 days. During the administration period, the rats were allowed to freely ingest a feed and water, and as the feed, the above-mentioned standard feed was used.

The day when the administration started was taken as day 0. On days 28, 49, and 63, blood was taken from the subclavian vein of the rats, and the blood was centrifuged to obtain a supernatant. The supernatant was used to measure the active OC content and the inactive OC content in the blood of the rats. The active OC content was measured using a kit for measuring active OC (Rat Gla-competitive EIA Kit: TAKARA SHUZO CO., LTD.). The inactive OC content was measured using a kit for measuring inactive OC (Rat Glu-competitive EIA Kit: TAKARA SHUZO CO., LTD.).

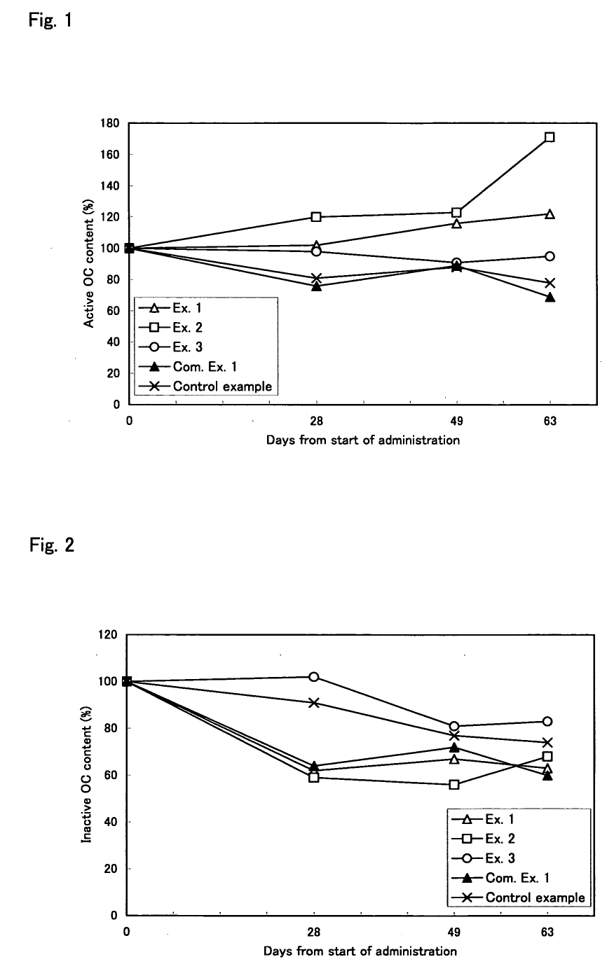

FIG. 1 shows an active OC content (%) measured at 28, 49, and 63 days after the start of the administration, wherein the active OC content on the day when the administration started is determined to be 100%. FIG. 2 shows an inactive OC content (%) measured at 28, 49, and 63 days after the start of the administration, wherein the inactive OC content on the day when the administration started is determined to be 100%. The average active OC content and the average inactive OC content on day 0 of the administration were 1.02 mg/mL and 0.65 mg/mL, respectively.

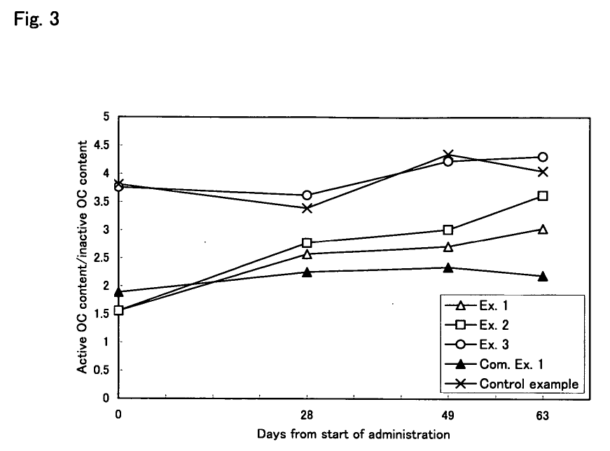

Furthermore, FIG. 3 shows a ratio (B/A) of the active OC content (B) to the inactive OC content (A) at 28, 49, and 63 days after the start of the administration. The lower the value of this ratio is, the more the bone resorption is promoted, and the more the balance of bone metabolism is disturbed.

### Example 2

The active OC content and the inactive OC content were measured in the same manner as in Example 1 except that a pine bark extract (trade name: Flavangenol, produced by TOYO SHINYAKU Co., Ltd.) containing 40 wt % of OPCs in terms of dry weight was used in place of the pine bark extract containing 5 wt % of OPCs in terms of dry weight. Thus, the active OC content (%), the inactive OC content (%), and the ratio (B/A) were obtained. FIGS. 1 to 3 show the results. The average active OC content and the average inactive OC content on day 0 of the administration were 1.00 mg/mL and 0.64 mg/mL, respectively.

### Example 3

The active OC content and the inactive OC content were measured in the same manner as in Example 2 except that the rats in the pseudo operation group were used in place of the rats in the spayed group. Thus, the active OC content (%), the inactive OC content (%), and the ratio (B/A) were obtained. FIGS. 1 to 3 show the results. The average active OC content and the average inactive OC content on day 0 of the administration were 2.02 mg/mL and 0.54 mg/mL, respectively.

### Comparative Example 1

The active OC content and the inactive OC content were measured in the same manner as in Example 1 except that water was used in place of the OPC-containing aqueous solution. Thus, the active OC content (%), the inactive OC content (%), and the ratio (B/A) were obtained. FIGS. 1 to 3 show the results. The average active OC content and the average inactive OC content on day 0 of the administration were 1.10 mg/mL and 0.59 mg/mL, respectively.

Moreover, a group of rats with the pseudo preration was provided as a control example, and the same procedure was conducted using these rats as in Comparative Example 1. The average active OC content (%) and the average inactive OC content (%) on day 0 of the administration were 2.14 mg/mL and 0.56 mg/mL, respectively.

As can be seen from FIG. 1, the active OC content tended to continually increase in the groups of spayed rats to which the OPC-containing aqueous solutions were administered (Examples 1 and 2) and tended to decrease in the group of spayed rats to which water was administered (Comparative Example 1) and the group of pseudo-operated rats to which water was administered (control example). This shows that the OPC-containing aqueous solutions provide an effect of promoting osteogenesis. In particular, in the group of spayed rats to which the aqueous solution that contained the pine bark extract containing 40 wt % of OPCs in terms of dry weight was administered (Example 2), the active OC content increased even more. On the other hand, in the group of pseudo-operated rats to which the OPC-containing aqueous solution was administered (Example 3), the active OC content did not increase. This shows that at a healthy level in which osteogenesis occurs adequately, osteogenesis does not promote further by the ingestion of OPCs. From the foregoing, it is shown that when an OPC-containing aqueous solution is administered, promotion of osteogenesis can be regulated as appropriate.

The active OC content in the serum (i.e., the supernatant) on day 63 of the administration was 1.67 mg/mL in the pseudo operation group in which water was administered (control group), while the active OC content was 1.71 mg/mL in the spayed group in which the OPC-containing aqueous solution was administered (Example 2). Thus, the active OC content in Example 2 was equal to or higher than that in the control example. This shows that the ability of osteogenesis that has been decreased due to menopausal disorders recovered to a healthy level through the administration of the OPC-containing aqueous solution. It should be noted that no difference in the weight was observed among the rats in all groups after the end of the administration, and also there was no significant difference in the amount of the feed each of the rats ingested.

As can be seen from FIG. 2, the inactive OC content showed the same tendency in all of Examples 1 to 3, Comparative Example 1, and the control example.

As can be seen from FIG. 3, the value of active OC content/inactive OC content increased steadily from a lower level in the groups in which the OPC-containing aqueous solutions were administered (Examples 1 and 2). On the other hand, the value consistently stayed at lower level in the group in which water was administered (Comparative Example 1). This shows that the OPC-containing aqueous solutions improved the balance of bone metabolism to a healthy condition. Furthermore, the value of the ratio of active OC content/inactive OC content on day 28 of the administration or later in the group in which the aqueous solution that contained the pine bark extract containing 40 wt % of OPCs in terms of dry weight was administered (Example 2) was in the range of about 70% to 90% of the value of the ratio of active OC content/inactive OC content in the group of pseudo-operated rats to which water was administered (control example). This shows that the balance of bone metabolism recovered significantly through the administration of the OPC-containing aqueous solution. The group of pseudo-operated rats to which the OPC-containing aqueous solution was administered (Example 3) showed almost the same tendency as in the group of pseudo-operated rats to which water was administered (control example). From the foregoing, it can be seen that through the administration of an OPC-containing aqueous solution, the balance of bone metabolism can be regulated to a healthy level.

The invention may be embodied in other forms without departing from the spirit or essential characteristics thereof. The embodiments disclosed in this specification are to be considered in all respects as illustrative and not limiting. The scope of the invention is indicated by the appended claims rather than by the foregoing description, and all changes which come within the meaning and range of equivalency of the claims are intended to be embraced therein.

1. A composition for regulating bone metabolism, comprising a proanthocyanidin.

2. The composition for regulating bone metabolism of claim 1, wherein the proanthocyanidin is contained in an extract derived from a natural product, and the extract comprises at least 20 wt % of oligomeric proanthocyanidins having a degree of polumerization of 2 to 4 in terms of dry weight.
